(19) United States
(12) Patent Application Publication
(10) Pub. No.: US 2005/0027127 A1
(43) Pub. Date: Feb. 3, 2005

(54) Oxadiazolyl-phenoxyalkylisoxazoles compositions thereof and methods for their use as anti-picornaviral agents

(76) Inventors: Guy D. Diana, Poltstom, PA (US); Theodore J. Nitz, Pottituwn, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/487,851

(22) PCT Filed: Aug. 29, 2002
(86) PCT No.: PCT/US02/27536

Related U.S. Application Data
(60) Provisional application No. 60/315,638, filed on Aug. 29, 2001.

(51) Int. Cl. C07D 413/02

(52) U.S. Cl. 548/243; 548/247

(57) ABSTRACT

Oxadiazolyl-phenoxyalkylisoxazoles and pharmaceutically acceptable salts thereof, compositions comprising oxadiazolyl-phenoxyalkylisoxazole compounds or pharmaceutically acceptable salts thereof and methods for using oxadiazolyl-phenoxyalkylisoxazole compounds or pharmaceutically acceptable salts thereof as anti-picornaviral agents are described herein.

### FIELD OF THE INVENTION

The present invention relates to oxadiazolyl-phenoxyalkylisoxazoles and pharmaceutically acceptable salts thereof, compositions comprising oxadiazolyl-phenoxyalkylisoxazole compounds or pharmaceutically acceptable salts thereof, and methods for using oxadiazolyl-phenoxyalkylisoxazole compounds or pharmaceutically acceptable salts thereof as anti-picomaviral agents.

### BACKGROUND OF THE INVENTION

Picornaviridae are a large group of single-stranded RNA viruses that cause infections such as viral meningitis, encephalitis, viral respiratory infection and viral exacerbation in asthma and colds (Picornavindae and Their Replication; Second Edition, edited by B. N. Fields et al., New York, 1990). Picornaviruses were among the first viruses recognized and encompass approximately 230 serotypes divided into five genera that include apthovirus, cardiovirus, enterovirus, hepatovirus and rhinovirus. Enterovirus and rhinovirus, which cause the common cold, comprise most of the known picornavirus serotypes.

The replication of single-stranded RNA viruses such as picornaviruses occurs entirely in the cytoplasm and has been well characterized. Briefly, the genome of picornaviruses consists of one single-stranded (+)-sense RNA molecule that encodes a single polyprotein, typically between about 2100 amino acids and about 2400 amino acids in length. The polyprotein is processed through proteolytic cleavage to provide viral proteins such as a picornavirus protease, a virus RNA-dependent RNA polymerase and various coat proteins. The cleavage of the polyprotein into the above proteins is highly specific and may provide an attractive target for pharmaceutical intervention. A significant problem in developing effective pharmaceutical treatment of picornaviral infections, however, is the rapid mutation rate of RNA viruses. This rapid mutation rate might be caused by the absence of any error-correcting mechanisms in RNA synthesis.

The cellular receptors of a number of different picornaviruses have been identified using a number of conventional techniques (for example, binding competition between different viruses, monoclonal antibodies that prevent virus binding and fluorescently labeled virus). Prevention of virus binding to cellular receptors is another attractive area for pharmaceutical intervention (Heinz et al., J. Virol., 1989, vol. 63, pp. 2476). Picornaviruses have also been characterized by X-ray crystallography (Rossman et al., Nature, 1985, 317, 145), which has been of great value in rationally designing inhibitors that interfere with virus-receptor binding.

Picornaviruses, particularly those of enterovirus and rhinovirus genera cause significant numbers of human viral infections each year. Effective therapies for the majority of picornaviral infections are inadequate or simply unavailable. Thus, there is a general need for agents active against picornaviruses and a specific need for agents active against enteroviruses and rhinoviruses.

U.S. Pat. No. 4,843,087 discloses diheterocylic compounds for use as anti-viral agents. U.S. Pat. Nos. 5,349,068 and 5,464,848 disclose 1,2,4-oxadiazolyl-phenoxyalkylisoxazoles and their use as anti-picornaviral agents.

Citation or identification of any reference in Section 2. of this Application is not an admission that such reference is available as prior art to the present invention.

### 3. SUMMARY OF THE INVENTION

The present invention provides compounds of Formula I: 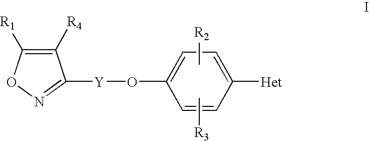
and pharmaceutically acceptable salts thereof wherein:
R₁ is selected from the group consisting of C₁-C₅ alkyl, C₁-C₅ alkoxy, C₃-C₇ cycloalkyl, C₁-C₅ alkoxycarbonyl, carboxy and cyanomethyl; the C₁-C₅ alkyl group being optionally substituted with at least one hydroxyl, C₁-C₅ alkoxyl, C₁-C₅ alkylthio, C₁-C₅ alkylsulfinyl, C₁-C₅ alkylsulfonyl, mono (C₁-C₅) alkylamino or di (C₁-C₅) alkyl amino groups and the C₁-C₅ alkoxy group being substituted with one or more hydroxyl groups;
Y is a straight or branched alkylene moiety of 3 to 9 carbon atoms;
R₂ and R₃ are independently selected from the group consisting of hydrogen, C₁-C₅ alkyl, C₁-C₅ alkoxy, halo, cyano, trifluoromethyl and nitro;
R₄ is selected from the group consisting of halo, hydrogen and C₁-C₅ alkyl; and

HET is selected from the group consisting of: 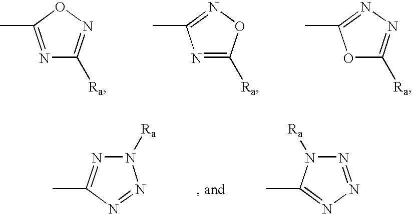
wherein Rₐ is selected from the group consisting of C₁-C₅ alkyl, C₁-C₅ alkoxy, C₃-C₇ cycloalkyl, heterocyclyl, C₁-C₅ alkoxycarbonyl, C₁-C₅ alkylthio, (4-methylphenyl)sulfonyloxymethyl, mono (C₁-C₅) alkylamino, di (C₁-C₅) alkylamino, and carboxamido; the C₁-C₅ alkyl group being optionally substituted with at least one halo, hydroxyl, C₁-C₅ alkoxyl, C₁-C₅ alkylcarbonyloxyl, thio and C₁-C₅ alkylthio groups.

The compounds of Formula I and pharmaceutically acceptable salts thereof are useful as anti-picornaviral agents.

In a second aspect, the present invention provides compositions comprising a therapeutically effective amount of a compound of Formula I or a pharmaceutically acceptable salt thereof. The compositions optionally comprise a pharmaceutically acceptable vehicle. The compositions are useful for treating or preventing a picomaviral infection in a patient.

In a third aspect, the present invention provides methods for treating or preventing a picornaviral infection in a patient, comprising administering to a patient in need of such treatment or prevention an effective amount of a compound of Formula I or a pharmaceutically acceptable salt thereof.

In a fourth aspect, the present invention provides methods for inhibiting the growth of a picornavirus in a cell comprising contacting a cell infected with a picornavirus with an effective amount of a compound of Formula I or a pharmaceutically acceptable salt thereof.

In a fifth aspect the invention provides a compound of Formula I, or a pharmaceutically acceptable salt thereof, in admixture with a compound of Formula II: 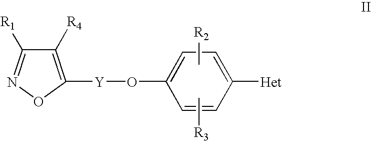
or a pharmaceutically acceptable salt thereof, wherein R₁, R₂, R₃, R₄, Y and HET are as previously defined in reference to the compound of Formula I.

In a sixth aspect, the invention provides compositions useful for treating or preventing a picornaviral infection in a patient, the compositions comprising a therapeutically effective amount of a compound of Formula I, or a pharmaceutically acceptable salt thereof, and a therapeutically effective compound of Formula U, or a pharmaceutically acceptable salt thereof, wherein R₁, R₂, R₃, R₄, Y and HET are as previously defined in reference to the compound of Formula I. The compositions optionally comprise a pharmaceutically acceptable vehicle.

In a seventh aspect, the invention provides methods for treating or preventing a picornaviral infection in a patient comprising administering to a patient in need of such treatment or prevention an effective amount of a combination of a compound of Formula I, or a pharmaceutically acceptable salt thereof, and a compound of Formula II, or a pharmaceutically acceptable salt thereof, wherein R₁, R₂, R₃, R₄, Y and BET are as previously defined in reference to the compound of Formula I.

In an eighth aspect, the invention provides methods for inhibiting growth of a picornavirus in a cell comprising contacting a cell infected with a picornavirus with an effective amount of a combination of a compound of Formula I, or a pharmaceutically acceptable salt thereof, and a compound of Formula II, or a pharmaceutically acceptable salt thereof, R₁, R₂, R₃, R₄, Y and HET are as previously defined in reference to the compound of Formula I.

### 4. DETAILED DESCRIPTION OF THE INVENTION

The present invention provides novel compounds having the general Formula I: 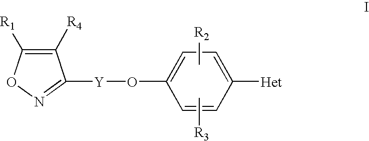
and pharmaceutically acceptable salts thereof wherein R₁, R₂, R₃, R₄, Y and HET are as defined above. The compounds of Formula I, and pharmaceutically acceptable salts thereof, are useful for treating or preventing a picornaviral infection in a patient and for inhibiting the growth of a picornavirus in a cell.

According to certain preferred embodiments, the compounds of Formula I are those having the Formulas III to VII below:

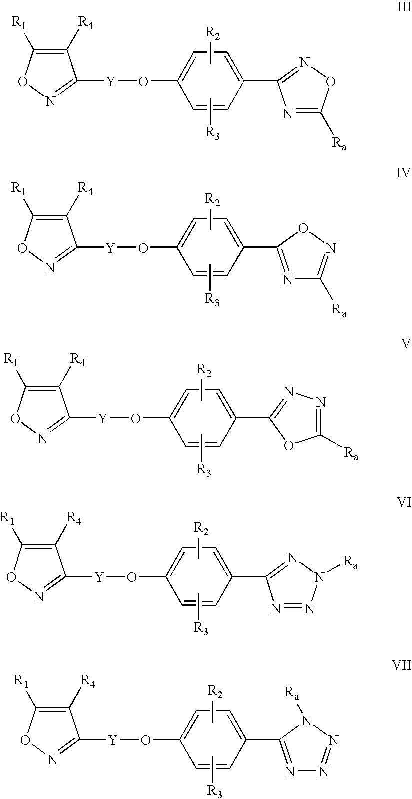

In all Formulas III to VII the groups R₁, R₂, R₃, R₄, Y and Rₐ are as defined above.

In other preferred embodiments, the compounds of Formula II are those having the Formulas VIII to XII, below:

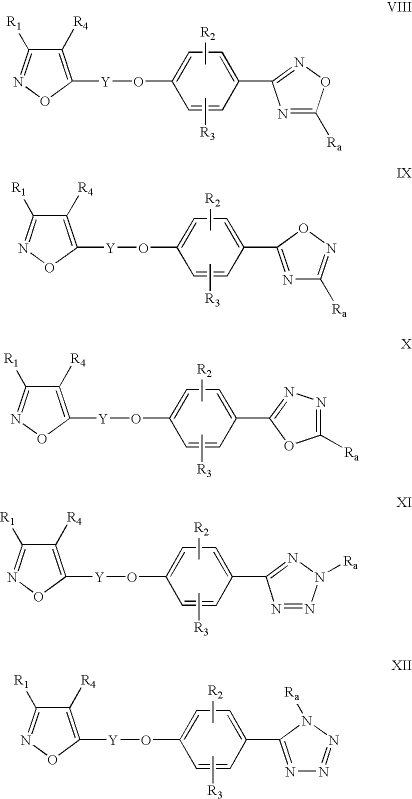

In all of Formulas VII to XII, R₁, R₂, R₃, R₄, Y and Rₐ are as defined above.

The compounds of Formulas I to XII and pharmaceutically acceptable salts thereof are useful as anti-picornaviral agents.

Preferred compounds of Formulas I to XII and pharmaceutically acceptable salts thereof are those wherein:

R₁ is selected from the group consisting of C₁-C₅ alkyl, C₁-C₅ alkoxy, cyclopropyl, hydroxy-C₁-C₅ alkyl, C₁-C₃ alkoxy-C₁-C₅-alkyl, hydroxy-C₁-C₅ alkoxy, methylthiomethyl, methylsulfinylomethyl, methylsulfonylmethyl and cyanomethyl;
Y is alkylene of 3 to 9 carbon atoms;
R₂ and R₃ are independently selected from the group consisting of hydrogen, C₁-C₃ alkyl, C₁-C₃ alkoxy, halo and cyano;
R₄ is selected from the group consisting of halo, hydrogen and C₁-C₃ alkyl; and

Rₐ is selected from the group consisting of C₁-C₃ alkyl, C₁-C₃ alkoxy, cyclopropyl, C₁-C₃ alkoxycarbonyl, and (4-methylphenyl)sulfonyloxymethyl; the C₁-C₃ alkyl group being substituted with one substituent selected from the group consisting of halo, hydroxyl, C₁-C₃ alkoxy or C₁-C₃ alkylcarbonyloxyl.

More preferred compounds of Formula I to XII and pharmaceutically acceptable salts thereof, are those wherein:
Y is alkylene of 3 to 5 carbon atoms;
R₁, R₂, R₃, R₄ and HET are as defined above; and Rₐ is selected from the group consisting of monohalomethyl; dihalomethyl; trihalomethyl; 1,1-dihaloethyl; 1,2-dihaloethyl; 2,2-dihaloethyl; 1,1,2-trihaloethyl; 1,2,2-trihaloethyl and 2,2,2-trihaloethyl.

In compounds wherein Rₐ is 2,2,2-trihaloethyl, Rₐ is preferably 2,2,2-trifluoroethyl.

Still more preferred compounds of Formula I and II to VII, and pharmaceutically acceptable salts thereof, are those wherein R₂ and R₃ are each in ortho position relative to the moiety:

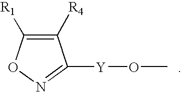

More preferred compounds of Formula II and VIII to XII and pharmaceutically acceptable salts thereof, are those wherein R₂ and R₃ are each in orthlo position relative to moiety:

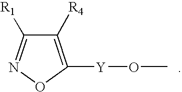

Most preferred compounds are: 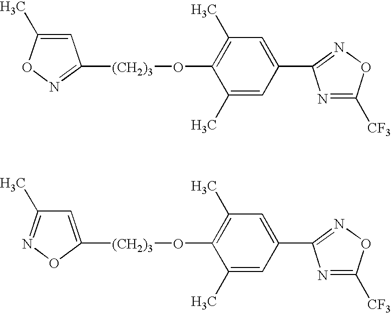
and pharmaceutically acceptable salts thereof.

### 4.1 Definitions and Abbreviations

As used herein, the term “compounds of the invention” means, collectively, the compounds of Formulas I to XII, pharmaceutically acceptable salts thereof, and mixtures thereof.

The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and, therefore, exist as stereoisomers, including enantiomers, and diastereomers, such as geometric isomers. According to the invention, the chemical structures depicted herein and therefore the compounds of the invention, encompass all of the corresponding compound's enantiomers and stereoisomers, that is, both the stereomerically pure form (for example, enantiomerically pure, or geometrically pure) and stereoisomeric mixtures. Enantiomeric and other stereoisomeric mixtures can be resolved into their components by well known methods, such as chiral-phase gas chromatography, chiral-phase high performance liquid chromatography, crystallizing the compound as a chiral salt complex, or crystallizing the compound in a chiral solvent. Enantiomers can also be obtained from enantiomerically-pure intermediates, reagents, and catalysts by well known asymmetric synthetic methods. The chemical structures depicted herein and therefore the compounds of the invention also encompass all of the corresponding compounds' possible tautomeric forms. Such tautomers may, in certain instances, be resolved into individual compounds by methods known to those of skill in the art.

When administered to a patient, for example, to an animal for veterinary use or for improvement of livestock, or to a human for clinical use, the compounds of the invention are preferentially administered in isolated form. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single oxadiazolyl-phenoxyalkylisoxazole compound of the invention by weight of the isolate.

The phrase “pharmaceutically acceptable salt(s),” as used herein includes but is not limited to salts of acidic or basic groups that may be present in compounds used in practicing the present invention. The anti-viral compounds described herein that are basic in nature are capable of forming a wide variety of salts with various inorganic and organic acids. The acids that may be used to prepare pharmaceutically acceptable acid addition salts of such basic compounds are those that form non-toxic acid addition salts, that is, salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (that is, 1,1′-methylene-bis-(2-hydroxy-3-naphthoate)) salts. Compounds included in the present compositions that include an amino moiety may form pharmaceutically acceptable salts with various amino acids, in addition to the acids mentioned above. The anti-viral compounds described herein that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “alkyl” means a saturated, monovalent unbranched or branched hydrocarbon chain. Examples of C₁-C₅ alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, and neopentyl.

As used herein, the term “cycloalkyl” means a monocyclic or polycyclic saturated ring comprising carbon and hydrogen atoms. Examples of C₃-C₇ cycloalkyl groups include, but are not limited to cyclopropyl, cyclobutyl, methylcyclobutyl, 2,3 diethyl cyclopentyl, cyclohexyl, and cycloheptyl.

As used herein, the term “alkoxy” means an —0-alkyl group, wherein alkyl is as defined above. Examples of C₁-C₅ alkoxy group include but are not limited to methoxy, ethoxy, propoxy, isopropoxy, 2-methyl-1-propoxy, 2-methyl-2-propoxy, 2-methyl-1-butoxy, 3-methyl-1-butoxy, 2-methyl-3-butoxy, 2,2-dimethyl-1-propoxy, 2-methyl-1-pentoxy, 3-methyl-1-pentoxy, 4-methyl-1-pentoxy, 2-methyl-2-pentoxy, 3-methyl-2-pentoxy, 4-methyl-2-pentoxy, 2,2-dimethyl-1-butoxy, 3,3-dimethyl-1-butoxy, 2-ethyl-1-butoxy, butoxy, isobutoxy, t-butoxy, pentoxy, isopentoxy, and neopentoxy.

As used herein the term “alkoxycarbonyl” means a monovalent group of the formula C(O)alkoxy. Examples of C₁₋₅ alkoxycarbonyl groups include but are not limited to methoxycarbonyl, ethoxycarbonyl, propoxycarbonyl, isopropoxycarbonyl, 2-methyl-1-propoxy carbonyl, 2-methyl-2-propoxycarbonyl, butoxycarbonyl, isobutoxycarbonyl and t-butoxycarbonyl.

As used herein, the term “alkylthio” means an —S-alkyl group, wherein alkyl is as defined above. Examples of C₁-C₅ thioalkyl groups include, but are not limited to, methylthio, ethylthio, propylthio, isopropylthio, 2-methyl-1-propylthio, 2-methyl-2-propylthio, 2-methyl-1-butylthio, 3-methyl-1-butylthio, 2-methyl-3-butylthio, 2,2-dimethyl-1-propylthio, 2-methyl-1-pentylthio, 3-methyl-1-pentylthio, 4-methyl-1-pentylthio, 2-methyl-2-pentylthio, 3-methyl-2-pentylthio, 4-methyl-2-pentylthio, 2,2-dimethyl-1-butylthio, 3,3-dimethyl-1-butylthio, 2-ethyl-1-butylthio, butylthio, isobutylthio, t-butylthio, pentylthio, isopentylthio, and neopentylthio.

As used herein, the term “alkylsulfinyl” means an O-alkyl group, wherein alkyl is as defined above. Examples of C₁-C₅ sulfinylalkyl groups include, but are not limited to, methylsulfinyl, ethylsulfinyl, propylsulfinyl, isopropylsulfinyl, 2-methyl-1-propylsulfinyl, 2-methyl-2-propylsulfinyl, 2-methyl-1-butylsulfinyl, 3-methyl-1-butylsulfinyl, 2-methyl-3-butylsulfinyl, 2,2-dimethyl-1-propylsulfinyl, 2-methyl-1-pentylsulfinyl, 3-methyl-1-pentylsulfinyl, 4-methyl-1-pentylsulfinyl, 2-methyl-2-pentylsulfinyl, 3-methyl-2-pentylsulfinyl, 4-methyl-2-pentylsulfinyl, 2,2-dimethyl-1-butylsulfinyl, 3,3-dimethyl-1-butylsulfinyl, 2-ethyl-1-butylsulfinyl, butylsulfinyl, isobutylsulfinyl, t-butylsulfinyl, pentylsulfinyl, isopentylsulfinyl, and neopentylsulfinyl.

As used herein, the term “alkylsulfonyl” means an —SO₂-alkyl group, wherein alkyl is as defined above. Examples of C₁-C₅ sulfonylalkyl groups include, but are not limited to, methylsulfonyl, ethylsulfonyl, propylsulfonyl, isopropylsulfonyl, 2-methyl-1-propylsulfonyl, 2-methyl-2-propylsulfonyl, 2-methyl-1-butylsulfonyl, 3-methyl-1-butylsulfonyl, 2-methyl-3-butylsulfonyl, 2,2-dimethyl-1-propylsulfonyl, 2-methyl-1-pentylsulfonyl, 3-methyl-1-pentylsulfonyl, 4-methyl-1-pentylsulfonyl, 2-methyl-2-pentylsulfonyl, 3-methyl-2-pentylsulfonyl, 4-methyl-2-pentylsulfonyl, 2,2-dimethyl-1-butylsulfonyl, 3,3-dimethyl-1-butylsulfonyl, 2-ethyl-1-butylsulfonyl, butylsulfonyl, isobutylsulfonyl, t-butylsulfonyl, pentylsulfonyl, isopentylsulfonyl, and neopentylsulfonyl.

As used herein, the term “monoalkylamino” means an —NH-alkyl group, wherein alkyl is as defined above. Examples of mono (C₁-C₅) alkylamino groups include but are not limited to methylamino, ethylamino, propylamino, isopropylamino, 2-methyl-1-propylamino, 2-methyl-2-propylamino, 2-methyl-1-butylamino, 3-methyl-1-butylamino, 2-methyl-3-butylamino, 2,2-dimethyl-1-propylamino, 2-methyl-1-pentylamino, 3-methyl-1-pentylamino, 4-methyl-1-pentylamino, 2-methyl-2-pentylamino, 3-methyl-2-pentylamino, 4-methyl-2-pentylamino, 2,2-dimethyl-1-butylamino, 3,3-dimethyl-1-butylamino, 2-ethyl-1-butylamino, butylamino, isobutylamino, t-butylamino, pentylamino, isopentylamino, and neopentylamino.

As used herein, the term “dialkylamino” means an N(alkyl)₂ group, wherein alkyl is as defined above. Examples of di (C₁-C₅) alkylamino groups include but are not limited to dimethylamino, diethylamino, dipropylamino, diisopropylamino, di-2-methyl-1-propylamino, di-2-methyl-2-propylamino, di-2-methyl-1-butylamino, di-3-methyl-1-butylamino, di-2-methyl-3-butylamino, di-2,2-dimethyl-1-propylamino, di-2-methyl-1-pentylamino, di-3-methyl-1-pentylamino, di-4-methyl-1-pentylamino, di-2-methyl-2-pentylamino, di-3-methyl-2-pentylamino, di-4-methyl-2-pentylamino, di-2,2-dimethyl-1-butylamino, di-3,3-dimethyl-1-butylamino, di-2-ethyl-1-butylamino, di-butylamino, di-isobutylamino, di-t-butylamino, di-pentylamino, di-isopentylamino and di-neopentylamino.

As used herein, the term “carboxamido” means a group of the formula —C(O)—NR′R″, wherein R′ and R″ represent hydrogen or an alkyl group.

As used herein, the term “alkylcarbonyloxyl” means a group of the formula R′—C(O)—O—, wherein R′ represents hydrogen or an alkyl group.

As used herein, the term “heterocyclyl” means an unsubstituted or substituted 5 or 6 membered heterocyclic ring, having from 1 to 4 (preferably 3 or 4) heteroatoms, for example, nitrogen, oxygen, sulfur or the like, the remainder of the ring atoms being carbon, and provided that no two oxygen and/or sulfur atoms are adjacent in the heterocyclyl group. Examples include furyl, thienyl, pyridyl, oxadiazolyl, thiadiazolyl, triazinyl and pyrimidinyl. Suitable heterocyclic ring substituents include C₁-C₅ alkyl and halo.

As used herein, the term “halo” means bromo, chloro, iodo or fluoro.

Examples of picornaviruses include but are not limited to enteroviruses, echoviruses, coxsackie virus and rhinovirus. Human rhinovirus serotypes (“HRV”) include, but are not limited to, HRV-2, -14, -1A, -1B, -6, -21, -22, -15, -25, -30, -50, -67, -89, -86 and 41 and HRV-3, -4, -5, -9, -16, -18, -38, -66, -75 and -61.

### 4.2 Synthesis of the Compounds of the Invention

The compounds of the invention can be obtained via the synthetic methodology illustrated in Schemes 1 to 8. Starting materials useful for preparing the compounds of the invention and intermediates therefore, are commercially available or can be prepared by well-known synthetic methods.

Scheme 1

The compounds of Formula I wherein R₁ is alkyl, alkoxy, cycloalkyl or alkoxyalkyl, Y, R₂, R₃, and R₄, are as defined for the preferred compounds of Formula I, and Rₐ is halomethyl, dihalomethyl, trihalomethyl, cycloalkyl, alkoxycarbonyl, alkoxyalkyl, alkane-carbonyloxyalkyl or 2,2,2-trifluoroethyl, and Het is 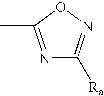
can be prepared by a process which involves reacting the amidoxime (N-hydroxycarboximidamide) of the formula: 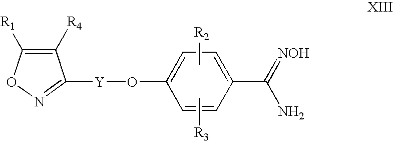
with an acid halide, RₐCOX or an acid anhydride, (RₐCO)₂O, where R₁, Y, R₂, R₃ and R₄ are as defined above in this paragraph and X is bromo, chloro, fluoro or iodo under anhydrous conditions to form the corresponding compound of Formula I. This process involves the following methods. In one method, the amidoxime XIII is reacted with the acid halide or the acid anhydride in the presence of an organic or inorganic base, for example, pyridine, triethylamine, or potassium carbonate; in an inert solvent, such as acetone, isopropylacetate, 1-methyl-2-pyrrolidinone, N,N-dimethylformamide (DMF), methylene chloride, chloroform, toluene, or tetrahydrofuran (THF); or in a base which also functions as the solvent, such as pyridine; at an elevated temperature (about 40° C. to 130° C.) or at a reduced temperature (about 0° C. to 15° C.). In the latter case an intermediate O-acyl derivative is isolated and heated at a temperature in the range of about 100° C. to 130° C., for a time sufficient for cyclization to the oxadiazole of Formula I to occur, generally about 5 minutes to 4 hours. In another method, the amidoxime is reacted with the acid halide or acid anhydride in an acid which corresponds to the acid halide or acid anhydride at an elevated temperature (about 70° C. to 100° C.).

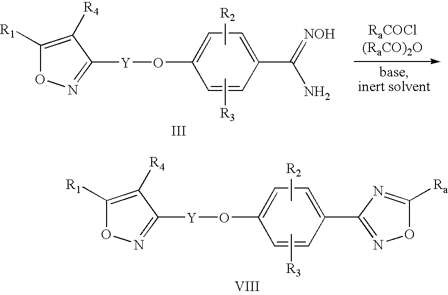

In another method, the amidoxime is reacted with an ester, RₐCO₂R_{b}, wherein Rₐ is CF₃ or CF₂CH₃ and R_{b} is a lower alkyl or phenyl, in N,N-dimethylformiamide or N-methylpyrrolidinone (NMP) as a co-solvent to aid in dissolution; at elevated temperatures (about 105° C.).

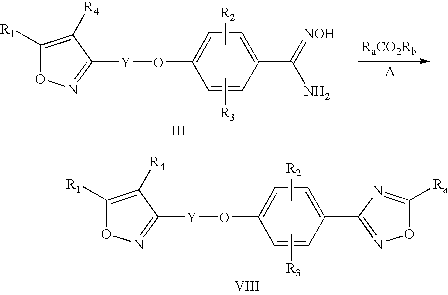

The compounds of Formula I where R₁ is alkyl, alkoxy, cycloalkyl, or alkoxyalkyl; Y, R₂, R₃, and R₄ are as defined hereinbefore, and Rₐ is dihalomethyl, trihalomethyl, cycloalkyl, alkoxyalkyl, alkylcarbonyloxyalkyl or 2,2,2-trifluoroethyl can be prepared by a process which comprises reacting the amidoxime XIII with the product obtained by the reaction of a carboxylic acid, RₐCO₂H, wherein R₁, Y, R₂, R₃, R₄ and Rₐ are defined as above in this paragraph, with the coupling agent N,N′-carbonyldiimidazole (CDI) for about thirty minutes to an hour; in an inert solvent, such as tetrahydrofuran, chloroform, methylene chloride, N,N-dimethylformamide, N-methylpyrrolidinone, or toluene; at an elevated temperature (about 80° C.) to form the corresponding compound of Formula I, without isolation of any intermediates.

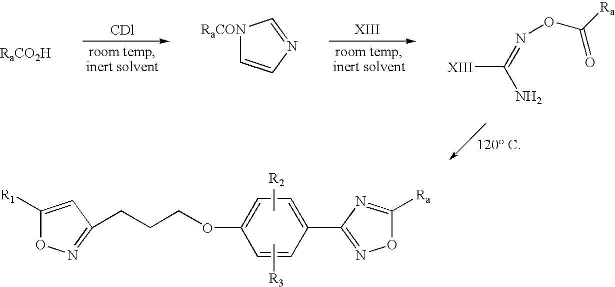

The compounds of Formula I where R₁ is alkyl, alkoxy, cycloalkyl, or alkoxyalkyl, Y, R₂, R₃ and Het are as defined hereinbefore and Rₐ is CH₂CF₃ and R₄ is hydrogen or alkyl, can be prepared by reacting the amidoxime XIII where R₁ is as defined above in this paragraph and Y, R₂, R₃ are as defined hereinbefore, with a ketene 1,3-propanedithiol acetal of the formula 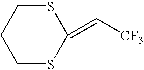
to give the corresponding compounds of Formula I. The amidoxime XIII and ketene 1,3-propanedithiol acetal are reacted in the presence of silver trifuoroacetate in an inert solvent, such as tetrahydrofuran, dioxane, dimethylformamide or N-methylpyrrolidinone; at a temperature in the range of from about 600 to about 100° C. Preferably the reaction is carried out in the dark.

The intermediate amidoxime XIII can be prepared in a step-wise manner by first reacting cyanophenol with a haloisoxazole, where X is chloro, bromo or iodo; in a dry inert solvent, such as N,N-dimethylformamide, acetonitrile, or N-methylpyrrolidinone; in the presence of base, such as potassium carbonate or sodium hydroxide; optionally in the presence of a catalytic amount of potassium or sodium iodide; at an elevated temperature (50° C. to 120° C.) to give cyano compound: 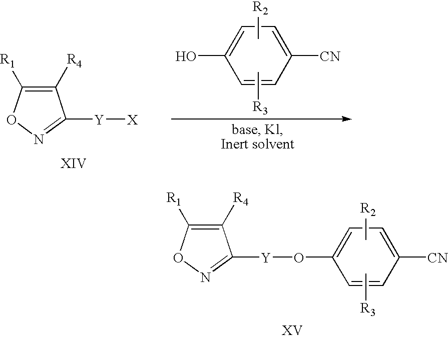
Then the cyano compound is reacted with hydroxylamine hydrochloride in the presence of a base, such as triethylamine (TEA), potassium or sodium carbonate, sodium acetate, or sodium alkoxide; in an alcoholic solvent, such as ethyl or butyl alcohol; at an elevated temperature (about 105° C.) to give the amidoxime XIII: 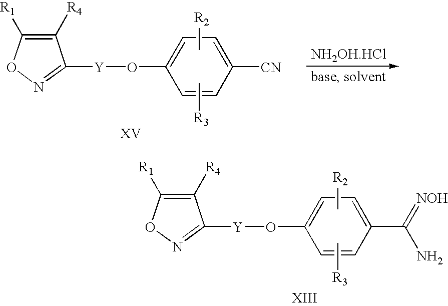
Scheme 2
The intermediate haloisoxazole (XIV) can be prepared using several methods.

(a) When Y is —(CH₂)₃—, and R₁ and R₄ are as defined hereinbefore, then haloisoxazole XIV can be prepared by first reacting the carboxylate of Formula XVI, where R_{c} is methyl, ethyl, propyl, or isopropyl, with sodium borohydride; in an alcoholic solvent, such as ethanol, to give the alcohol of Formula XVII. The intermediate isoxazole methanols of Formula XVII can then be converted to the bromide of Formula XVII by a variety of methods that include triphenylphosphine in conjunction with bromine, N-bromosuccinimide (NBS), or CBr₄, or with phosphorous tribromide, phosphorous pentabromide, phosphorous oxybromide, or thionyl bromide in an inert solvent, such as tetrahydrofuran, chloroform, methylene chloride, dichloroethane, dioxane, or toluene; than slowly adding the alcohol of Formula XVII. 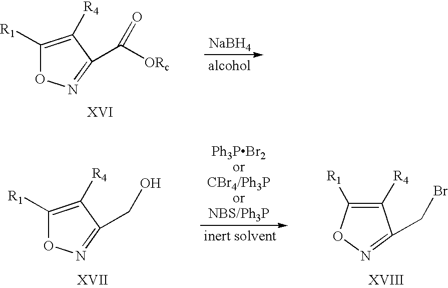
Treatment of the bromides of Formula XVIII in the presence of hexamethylphosphoramide (HMPA) with the anion of tert-butyl acetate, prepared from tert-butyl acetate and lithium diisopropylamide (LDA), lithium ispropylcyclohexylamide (LICA), or lithium diethylamide (LDEA); in an inert solvent such as tetrahydrofuran (THF), provided the tert-butyl ester of Formula XIX, which was reduced with lithium aluminum hydride (LAH) in an inert solvent, such as diethyl ether, to give the intermediate alcohol of Formula XX. 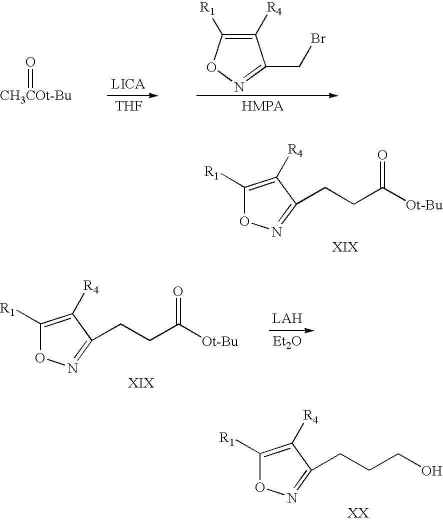
(b) When Y is —(CH₂)₅—, and R₁ and R₄ are as defined hereinbefore, then the haloisoxazole XIV can be prepared by one of the following reactions schemes: 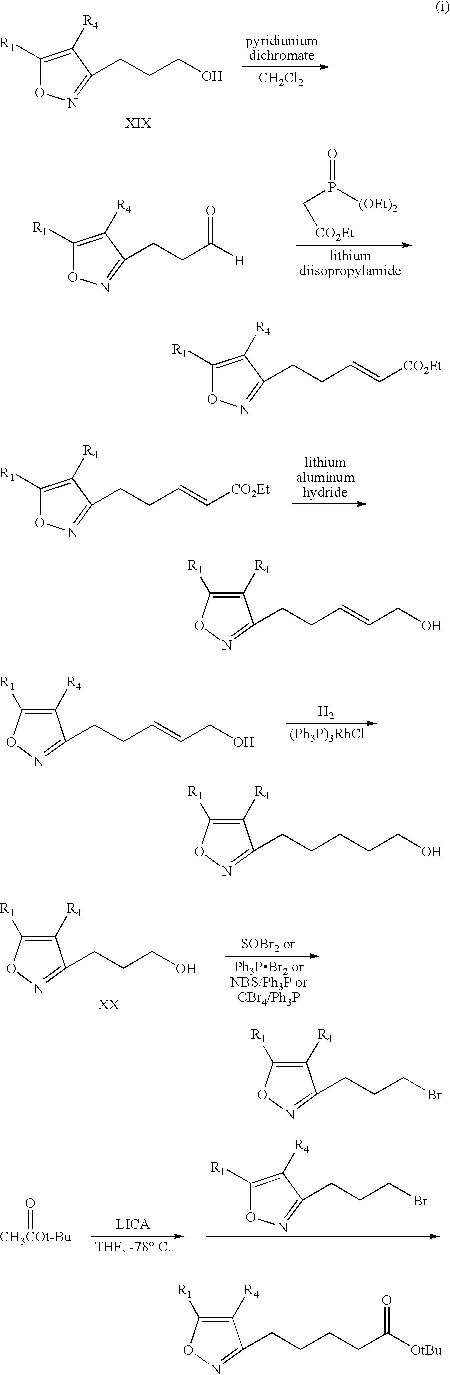 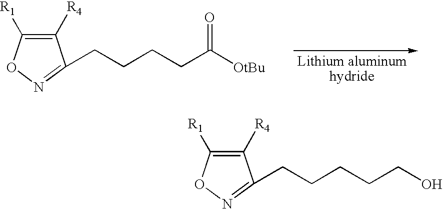
   Note, the reagent PDC denotes “pyridinium dichromate.” For each of these methods, the alcohol can than be converted to the intermediate haloisoxazole of Formula XIV as previously shown, with triphenylphosphine (PH₃P) and a halogenating agent, such as chlorine, bromine, N-chloro or N-bromosuccinimde (NBS) or carbon tetrachloride, or with thionyl chloride, thionyl bromide, phosphorus chloride or phosphorus bromide. 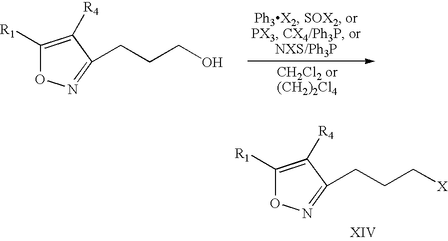
   Scheme 3

The intermediate carboxylate of Formula XVI can be prepared using the following methods. The carboxylate intermediate of Formula XVI for compounds of Formula I where R₁ is alkyl, cycloalkyl, or alkoxyalkyl, R₄ is hydrogen and R_{C} is methyl, ethyl, propyl, or isopropyl, can be prepared according to the following process: 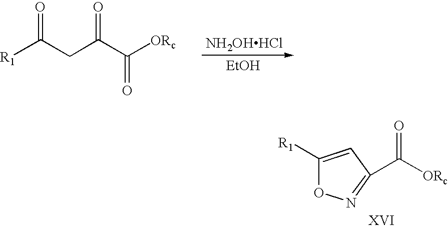
or for compounds where Rₐ is as defined above in this paragraph, and R₄ is alkyl or halogen according to the following process: 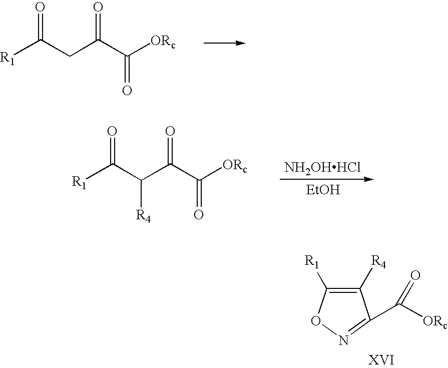
The carboxylate intermediate of Formula XVI for compounds of Formula I where R₁ is alkyl, cycloalkyl, or alkoxyalkyl, can be prepared according to the following process: 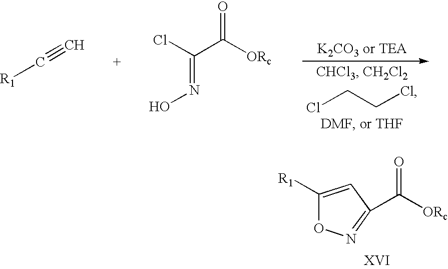
The carboxylate intermediate of Formula XVI for compounds of Formula I where R₁ is alkyl, cycloalkyl, or alkoxyalkyl, can be prepared according to the following process: 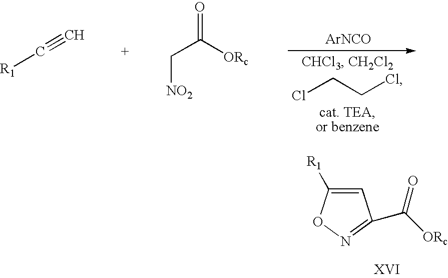
The carboxylate intermediate of Formula XVI for compounds of Formula I where R₁ is hydroxymethyl or methylthio and R′, R″, and R′″ are lower alkyl or phenyl, can be prepared according to the following process: 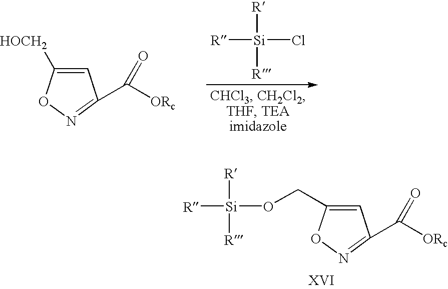
Scheme 4

The compounds of Formula I wherein R₁ is hydroxyalkyl, Y, R₂, R₃ Het and Rₐ, are as defined for the preferred compounds of Formula I, and R₄ is hydrogen or an alkyl group can be prepared from a compound of the formula: 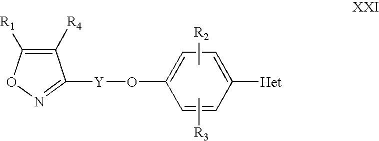
wherein R₁′ is tert-butyldimethylsilyloxyalkyl ((CH₃)₃CSi(Me)₂—O-alkyl) and Y, R₁, R₃, Het and R₄ are as defined above in this paragraph, by cleaving the tert-butyldimethylsilyl ether.

Cleavage of the tert-butyldimethylsilyl ether is carried out by treating compound XXI as defined above, with organic acids, for example, acetic acid or trifluoroacetic acid, or inorganic acid, such as hydrochloric acid or sulfuric acid, or using a source of fluoride, such as tetrabutylammonium fluoride (TBAF); in an inert solvent, such as tetrahydrofuran (THF) or dioxane; in the presence of water at a temperature in the range from about 20° to about 60° C.

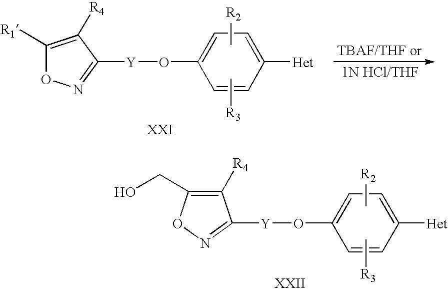

The compound of Formula XXI can be prepared by a process which comprises reacting a phenol Formula XXIII wherein R₂, R₃, Het and Rₐ are as defined hereinbefore, R₄ is hydrogen or lower alkyl, with an isoxazole of the Formula XXIV according to the following process:

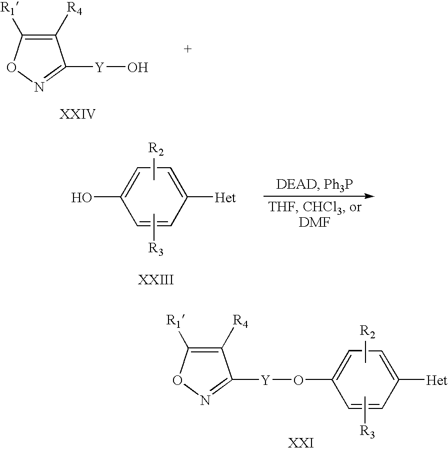

The phenol of Formula XXIII is reacted with isoxazole Formula XXIV in the presence of diethyl azodicarboxylate (DEAD) and triphenylphosphine in an inert solvent, such as tetrahydrofuran, chloroform, dimethylformamide or N-methylpyrrolidinone; at a temperature in the range of from about −20° C. to about 20° C.

Several methods can be used to synthesize the intermediate phenol Formula XXIII.

(a) In one method, the phenol XII, where R₂ and R₃ are as defined for the preferred compounds of Formula I, can be prepared by reacting cyanophenol with hydroxylamine hydrochloride, using a procedure similar to that described hereinbefore for the preparation of amidoxime XIII from the cyano compound, to give an amidoxime of the formula:

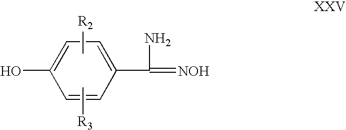

Amidoxime XXV is reacted with RₐCOX, (RₐCO)₂O, RₐCO₂H or 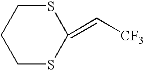
using procedures similar to those described hereinbefore for the preparation of Formula I from amidoxime XIII, to give the corresponding phenol.

(b) According to another method, the intermediate phenol XXIII, where R₂ and R₃ are as defined for the preferred compounds of Formula I, can be prepared by treating the nitrile XXVI with sodium azide to provide tetrazole XXVII: 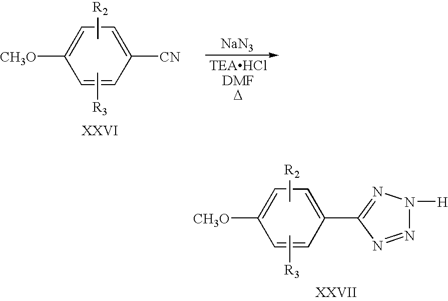
The tetrazole XXVII is converted into the oxadiazole by acylation of the tetrazole with acid chloride or acid anhydride, followed by thermolysis to intermediate XXVIII. 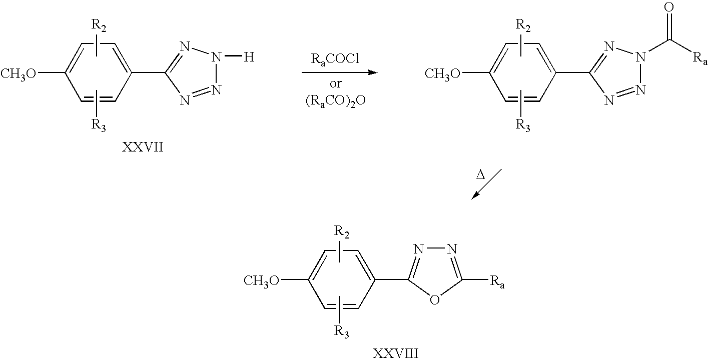
Treatment with boron tribromide gives the intermediate phenol of Formula XX. 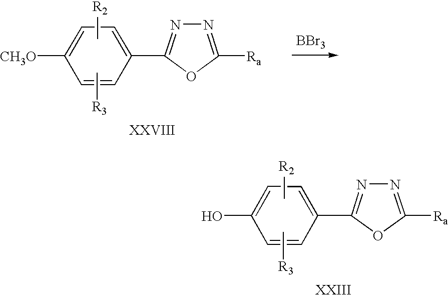
(c) Another method to make the intermediate phenol XXIII, where R₂ and R₃ are as defined for the preferred compounds of Formula IV, involves O-acylation of an amidoxime and thermalization to XXIX. 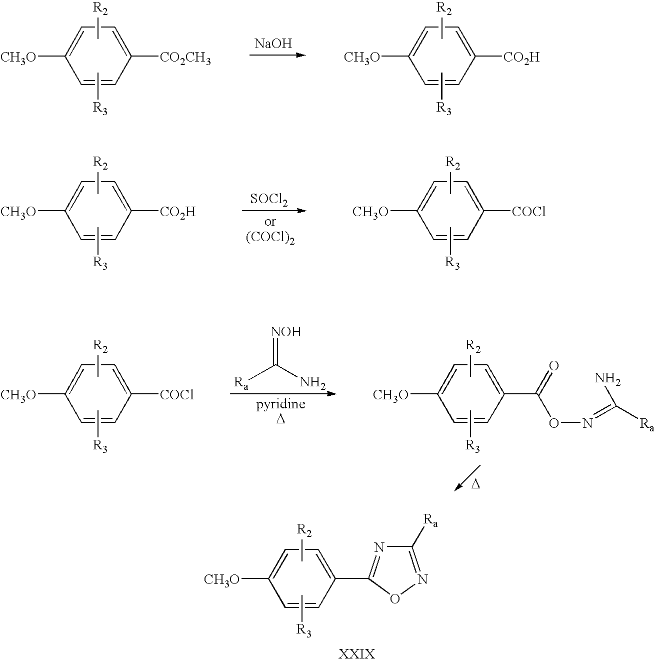
Treatment with borontribromide provides the intermediate phenol XXIII: 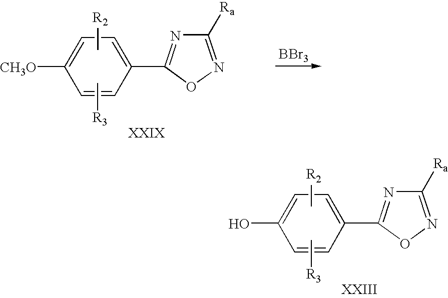
Scheme 5

The compounds of Formula I where R₁ is CH₃SCH₂, CH₃SOCH₂, and CH₃SO₂CH₂; R₂, R₃, Y, and Het are as defined hereinbefore and Rₐ is dihalomethyl, trihalomethyl, cycloalkyl, alkoxyalkyl, or 2,2,2-trifluoroethyl and R₄ is hydrogen or lower alkyl, can be prepared by reactions using alcohol XXII. The alcohol is reacted with the reagent prepared from dimethyldisulfide and triphenylphosphine to give the thio ether. 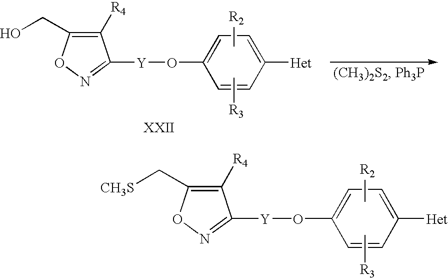
The thio ether compound is oxidized with oxone in the presence of wet alumina resulting in a mixture of separable sulfoxide and sulfone. 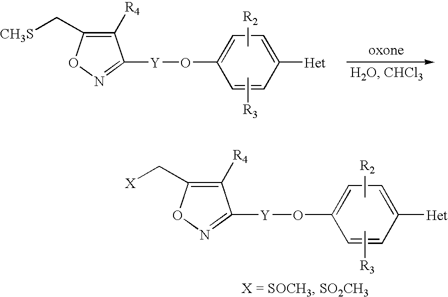
Scheme 6
Compounds of the Formula: 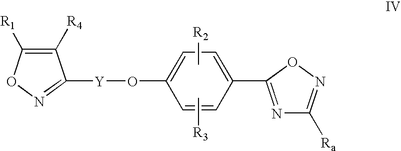
wherein R₁, Y, R₂, R₃, and R₄, are as defined for the preferred compounds of Formula I, and Rₐ is alkyl, halomethyl, dihalomethyl, trihalomethyl, cycloalkyl, or alkoxyalkyl, can be prepared according to the following scheme: 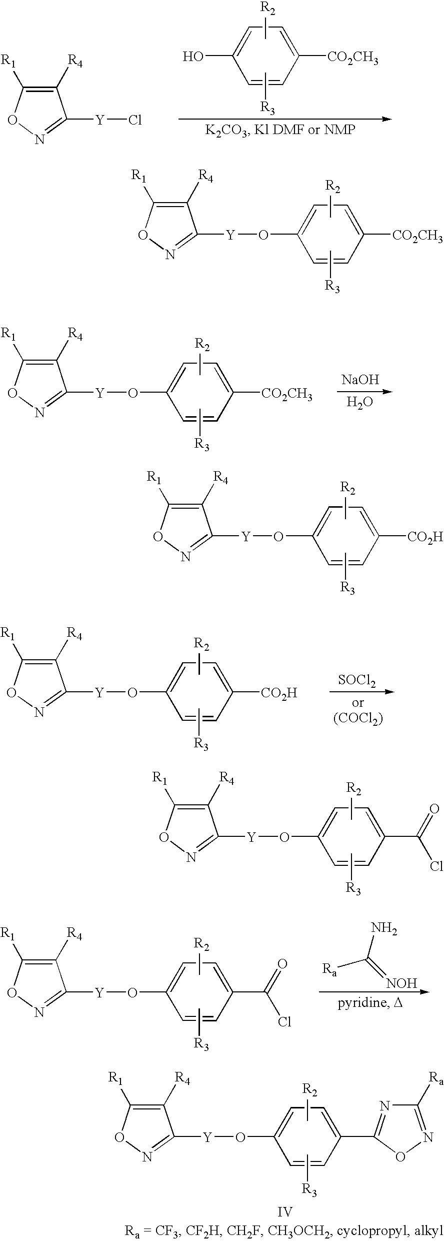
The hydroxybenzoate is caused to react with a haloalkylisoxazole to give the ester, which is hydrolyzed to the corresponding acid. The latter is then converted to its acid chloride which is reacted with an amidoxime to give the acylamidoxime, which is thermally cyclized to give compounds of the Formula IV.
Scheme 7

Compounds of the formula: 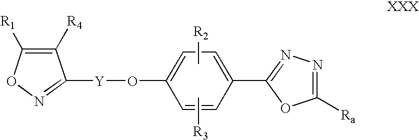
where R₁ is alkyl, alkoxy, cycloalkyl, or alkoxyalkyl, Y, R₂, and R₃ are as defined for the preferred compounds of Formula I, and Rₐ is alkyl, cycloalkyl, halomethyl, dihalomethyl, trihalomethyl, alkoxyalkyl, or 2,2,2-trifluoroethyl and R₄ is hydrogen or lower alkyl can be prepared by reacting the cyano compound XV with sodium azide to give tetrazole XXXI.

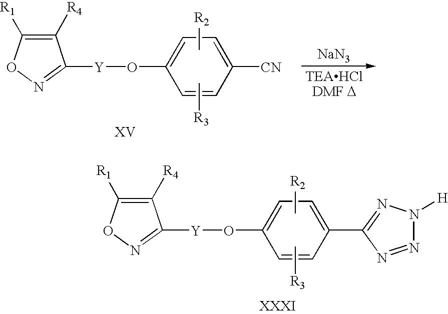

N-Acylation of XXXI, followed by thermal cyclization and loss of nitrogen provides compounds of the Formula XXX. 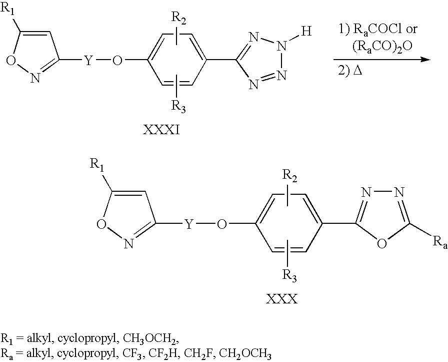
Scheme 8
Compounds of the Formula: 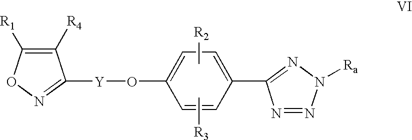
where R₁ is alkyl, alkoxy, cycloalkyl, or alkoxyalkyl, Y, R₂, R₃, and R₄ are as defined for the preferred compounds of Formula I, and Rₐ is alkyl, cycloalkyl, alkoxyalkyl, or 2,2,2-trifluoroethyl and R₅ is hydrogen or lower alkyl can be prepared by treating tetrazole XXXI with alkylhalide, which gives a mixture of 1- and 2-substituted tetrazoles, which can be separated by high-pressure liquid chromatography (HPLC).

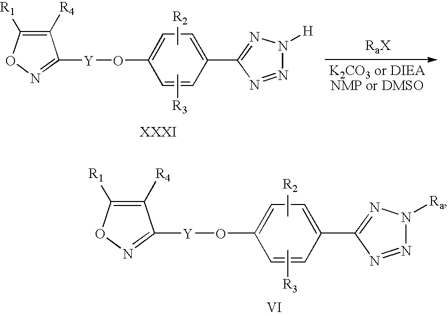

### 4.3 Therapeutic Uses of the Compounds of the Invention

In carrying out the method of the invention in its broadest aspect, a compound of the invention is administered to a patient, preferably a human, infected with a picornaviral infection. In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, for example, stabilization of a discernible symptom, physiologically, for example, stabilization of a physical parameter, or both. In yet another embodiment, “treatment” or “treating” refers to delaying the onset of a disease or disorder.

In certain embodiments, the compounds of the invention are administered prophylactically to a patient, preferably a human, as a preventative measure against such diseases. As used herein, “prevention” or “preventing” refers to a reduction of the risk of acquiring a given disease or disorder. In a preferred mode of the embodiment, the compositions of the invention are administered as a preventative measure to a patient, preferably a human, having a predisposition to a picornaviral infection.

### 4.4 Therapeutic/Prophylactic Administration and Compositions

Due to the activity of the compounds of the invention, the compounds are advantageously useful in veterinary and human medicine. As described above, the compounds of the invention are useful for the treatment or prevention of picornaviral infections.

The invention provides methods of treatment and prophylaxis by administration to a patient of a therapeutically effective amount a compound of the invention. The patient is an animal, including, but not limited, to an animal such a cow, horse, sheep, pig, chicken, turkey, quail, cat, dog, mouse, rat, rabbit and guinea pig and is more preferably a mammal and most preferably a human.

The compositions of the invention, which comprise a compound of Formula I and optionally a pharmaceutically acceptable vehicle, are preferably administered orally. The compounds of the invention can be administered by any other convenient route, for example, by infusion or bolus injection, by absorption through epithelial or mucocutaneous linings (for example, oral mucosa, rectal and intestinal mucosa) and can be administered together with another biologically active agent. Administration can be systemic or local. Various delivery systems are known, for example, encapsulation in liposomes, microparticles, microcapsules and capsules and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. Modes of administration are left to the discretion of the practitioner and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, for example, in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce compounds of the invention into the central nervous system by any suitable route, including intraventricular, intrathecal and epidural injection. Intraventricular injection can be facilitated by an intraventricular catheter, for example, attached to a reservoir, such as an Ommaya reservoir.

Pulmonary administration can also be employed, for example, by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds of the invention can be delivered in a controlled-release system. In one embodiment, a pump can be used (see Langer, supra; Sefton, 1987, CRC Crit. Ref Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity to the target of the compounds of the invention, for example, the liver, thus requiring only a fraction of the systemic dose (see, for example, Goodson, in Medical Applications of Controlled Release, supra, vol. 2, pp. 115-138 (1984)). Other controlled-release systems discussed in the review by Langer, 1990, (Science 249:1527-1533) can be used.

The present compositions contain a therapeutically effective amount of a compound of the invention, optionally more than one compound of the invention, preferably in purified form, and optionally together with a suitable amount of a pharmaceutically acceptable vehicle so as to provide the form for proper administration to the patient.

In a specific embodiment, the term “pharmaceutically acceptable” means approved by a regulatory agency of the Federal or a state government, listed in the U.S. Pharmacopeia or other generally recognized pharmacopeia for use in animals and more particularly in humans; or generally regarded by those of skill in the art as being safe to a patient. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see for example, U.S. Pat. No. 5,698,155). Other examples of suitable pharmaceutical vehicles are described in “Remington's Pharmaceutical Sciences” Gennard A. R., (Ed.), Mack Publishing Co., Pennsylvania (1985).

In a preferred embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. Where necessary, the compositions may also include a solubilizing agent. Compositions for intravenous administration may optionally include a local anesthetic such as lidocaine to ease pain at the site of the injection. Generally, the components of the present compositions are supplied either separately or mixed together in unit dosage form, for example, as a dry lyophilized powder or water free concentrate in a hermetically sealed container such as an ampoule or sachette indicating the quantity of active agent. Where a compound of the invention is to be administered by infusion, it can be dispensed, for example, with an infusion bottle containing sterile pharmaceutical-grade water or saline. Where a compound of the invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard vehicles such as mannitol, lactose, starch, magnesium stearate, sodium saccharin, cellulose and magnesium carbonate. Such vehicles are preferably of pharmaceutical grade.

The amount of a compound of the invention that will be effective in the treatment of a picomaviral infection will depend on the nature of the infection and can be determined by standard clinical techniques. In addition, in vitro or in vivo assays can optionally be employed to help identify optimal dosage ranges. The precise dose to be employed will also depend on the route of administration and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In specific preferred embodiments of the invention, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for picornaviral infection. The compounds of the invention may also be demonstrated to be effective and safe using animal model systems.

Because compounds within the scope of the invention are able to suppress the growth of viruses when added to a medium in which the virus is growing, it is specifically contemplated that compounds of the invention can therefore be used in aqueous solution, optionally, in the presence of a surfactant, to decontaminate surfaces on which viruses, including polio and picornaviruses are present, such surfaces including, but not limited to, hospital glassware, hospital working surfaces and similar areas in the preparation of food.

Hand contact of nasal mucus is an important mode of picomaviral, particularly, rhinoviral transmission. Disinfection and/or sterilization of the hands of people coming into contact with persons infected with a picornavirus would be a method useful for preventing further spread of the disease. If a compound of the invention were incorporated into a hand-washing procedure or hand-care composition, such procedure or composition may inhibit replication of picornaviruses and decrease the likelihood of the transmission of the disease.

Where a compound of the invention is administered prior to infection, that is, prophylactically, it is preferred that the administration be performed within 0 to 48 hours prior to infection of the host animal with the pathogenic virus. Where a compound of the invention is administered therapeutically to inhibit replication of a picornavirus or progression of infection, it is preferred that the administration be performed within about a day or two after infection with the pathogenic virus.

Other methods of use of the compounds described herein will be known to the skilled artisan and are within the scope of the invention.

### 4.5 Combination Therapy

In certain embodiments of the present invention, the compounds of the invention can be used in combination therapy with at least one other therapeutic agent. The compound of the invention and the therapeutic agent can act additively or, more preferably, synergistically. In a preferred embodiment, a compound of the invention is administered concurrently with the administration of another therapeutic agent, which can be administered as a component of a composition comprising the compound of the invention or as a component of a different composition. In another embodiment, a composition comprising a compound of the invention is administered prior or subsequent to administration of another therapeutic agent. In certain embodiments, where a compound of the invention is administered in combination with another therapeutic agent that potentially produces adverse side effects including but not limited to toxicity, the therapeutic agent can advantageously be administered at a dose that falls below the threshold at which the adverse side is elicited. Preferably, the therapeutic agent is an antiviral or antibacterial agent. The present compositions can be administered together with a known drug.

### 5. Example

### 3-[3,5-Dimethyl-4-[3-(5-methyl-3-isoxazoyl)propoxy]phenyl]-5-trifluoromethyl-1,2,4-oxadiazole

(a) Ethyl 5-methylisoxazole-3-carboxylate: To an absolute ethanol (3.2 L) solution of ethyl 2,4-dioxovalerate (200 g, 1.27 mol) was added hydroxylamine hydrochloride (308 g, 4.44 mol). After heating at reflux for 5 hours, the reaction solution was cooled and the volatiles removed in vacuo. The residual oil was partitioned between water (1 L) and t-butyl methyl ether (500 ml) and separated. The aqueous phase was extracted with t-butyl methyl ether (2×250 ml). The combined organic phases were washed with 5% sodium hydroxide (3×100 ml), water, and brine, then dried (MgSO₄) and filtered through a short column of Florisil, eluting with an additional 100 ml of t-butyl methyl ether. Concentration of the filtrate in vacuo provided 123 g of product as a yellow oil.

(b) 5-Methyl-3-isoxazolemethanol: To an ice cold absolute ethanol (1.6 L) solution of the above ester (123 g, 0.792 mol) was added NaBH₄ (59.9 g, 1058 mol) in about 5 g portions over 15 minutes. The ice bath was removed. After 3 hours at room temperature, the reaction solution was rechilled and treated dropwise with concentrated hydrochloric acid (115 ml). After 14 hours at room temperature, the pH of the mixture was adjusted to about pH 6 with 15% sodium hydroxide, then filtered. The filter cake was washed with absolute ethanol (2×100 ml). The combined filtrates were concentrated in vacuo. The residual was partitioned between ethyl acetate (250 ml) and sufficient water to dissolve the salts. After saturating with (NH₄)₂SO₄, the organic phase was separated and the aqueous phase extracted with ethyl acetate (3×100 ml). The combined organic phases were washed with brine saturated with (NH₄)₂SO₄, dried (MgSO₄), and concentrated in vacuo. Distillation of the residual oil provided 66.9 g of product as a colorless oil with a boiling point of 122-125° C. at 12 mm Hg.

(c) 3-Bromomethyl-5-methylisoxazole: A solution of bromine (34.0 ml, 0.660 g-atom) in methylene chloride (600 ml) was added dropwise to an ice-cold solution of triphenylphosphine (173 g, 0.660 mol) in methylene chloride (1.2 L) at a rate to maintain reaction temperature less than or equal to 4° C. After 15 minutes, a solution of 5-methyl-3-isoxazolemethanol (67.9 g, 0.600 mol) in methylene chloride (600 ml) was added over 2 hours. After 1 hour, the reaction was diluted with hexanes (2.5 L), chilled at −25° C. for 14 hours, and filtered. The filtrate was concentrated in vacuo. The residual yellow paste (235 g) was suspended in hexanes (500 ml) and filtered. The filter cake was washed with hexanes (3×100 ml). The combined filtrates were concentrated in vacuo to give a yellow oil (103 g) that was filtered through a column of Silica Gel 60 (150 g) with hexanes (1 L). Concentration in vacuo gave 78.3 g of product as a pale yellow oil.

(d) t-Butyl 5-methyl-3-isoxazolepropionate: n-Butyllithium (100 ml, 2.5 M in hexanes) was added over 1 hour to a tetrahydrofuran (1 L) solution of N-isopropylcyclohexylamine (41.9 ml, 0.255 mol) chilled to −78° C. After 15 minutes, t-butyl acetate (34.4 ml, 0.255 mol) was added dropwise over 30 minutes. After 1 hour, a solution of 3-bromomethyl-5-methylisoxazole (39.1 g, 0.220 mol) in hexamethylphosphoramide (58 ml, 0.33 mol) was added dropwise over 2 hours, stirred an additional 2 hours, treated with 10% NH₄Cl (250 ml), and warmed to room temperature. The biphasic mixture was partitioned between water (1 L) and t-butyl methyl ether (250 ml). The organic phase was separated, and the aqueous phase extracted with t-butyl methyl ether (250 ml). The combined organic phases were washed with 1 N hydrochloric acid (2×250 ml) and brine, dried with MgSO₄, and concentrated in vacuo to give a yellow oil (47.8 g). Distillation gave 34.3 g of product as a colorless oil, with a boiling point of 86-90° C. at 0.2 mm Hg.

(e) 5-Methyl-3-isoxazolepropanol: A suspension of lithium aluminum hydride (4.86 g, 0.128 mol) in ethyl ether (160 ml) was chilled at 0° C. A solution of t-butyl 5-methyl-3-isoxazolepropionate (33.8 g, 0.160 mol) in ethyl ether (160 ml) was added dropwise over 2 hours, stirred for 1 hour, and treated sequentially with water (4.8 ml), 15% sodium hydroxide (4.8 ml), and water (14.7 ml). The mixture was then dried with K₂CO₃, filtered through a short column of Florisil, and concentrated in vacuo. Distillation of the obtained oil (23.3 g) provided 19.7 g of product as a colorless oil with a boiling point of 86-90° C. at 0.2 mmHg.

(f) 3-[3,5-Dimethyl-4-[3(5-methyl-3-isoxazoyl)propoxy]phenyl]-5-trifluoromethyl-1,2,4-oxadiazole: A suspension of 2,6-dimethyl-4-[5-(trifluoromethyl)-1,2,4-oxadiazol-3-yl]phenol (23.2 g, 90.0 mmol), methylene chloride (225 ml), 5-methyl-3-isoxazolepropanol (15.2 g, 108 mmol), and triphenylphosphine (28.3 g, 108 mmol) was chilled with an ice bath. A solution of diethyl azodicarboxylate (17.0 ml, 108 mmol) in methylene chloride (90 ml) was added dropwise over 90 minutes. The reaction was stirred at room temperature for 14 hours, then concentrated in vacuo to give a solid (85.7 g). The solid was dissolved into warm toluene (100 ml) and applied to a column of Silica Gel 60 (500 g) which was prepacked in toluene. Elution with toluene (2 L), followed by 2.5% ethyl acetate in toluene, provided 32.4 g of product as a colorless oil that solidified upon standing. Crystallization from methanol provided product as white needles with a melting point of 50-51° C.

The present invention is not to be limited in scope by the specific embodiments disclosed in the examples which are intended as illustrations of a few aspects of the invention. Any embodiments which are functionally equivalent are within the scope of this invention. Indeed, various modifications of the invention in addition to those shown and described herein will become apparent to those skilled in the art and are intended to fall within the scope of the appended claims.

A number of references have been cited in the foregoing specification, the entire disclosures of which are incorporated by reference hererin.

1. A compound of Formula I: 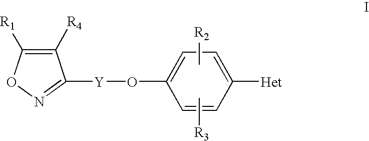
and pharmaceutically acceptable salts thereof wherein:
R₁ is selected from the group consisting of C₁-C₅ alkyl, C₁-C₅ alkoxy, C₃-C₇ cycloalkyl, C₁-C₅ alkoxycarbonyl, carboxy and cyanomethyl the C₁-C₅ alkyl group being optionally substituted with at least one hydroxyl. C₁-C₅ alkoxyl, C₁-C₅ alkylthio, Cts alkylsulfinyl, C₁-C₅ alkylsulfonyl, mono (C₁-C₅) alkylamino or di (C₁-C₅) alkylamino groups and the C₁-C₅ alkoxy group being substituted with one or more hydroxyl groups;
Y is a straight or branched alkylene moiety of 3 to 9 carbon atoms;
R₂ and R₃ are independently selected from the group consisting of hydrogen, C₁-C₅ alkyl, C₁-C₅ alkoxy, halo, cyano, trifluoromethyl and nitro;
R₄ is selected from the group consisting of halo, hydrogen and C₁-C₅ alkyl; and BET is selected from the group consisting of: 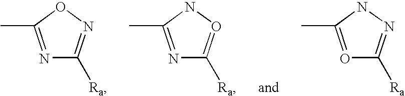
wherein Rₐ is selected from the group consisting of C₁-C₅ alkyl, C₁-C₅ alkoxy, C₃-C₇ cycloalkyl, heterocyclyl, C₁-C₅ alkoxycarbonyl. C₁-C₅ alkylthio, (4-methylphenyl)sulfonyloxymethyl, mono (C₁-C₅) alkylamino di (C₁-C₅) alkylamino, and carboxamido; the C₁-C₅ alkyl group being optionally substituted with at least one halo, hydroxyl, C₁-C₅ alkoxyl, C₁-C₅ alkylcarbonyloxyl, thio and C₁-C₅ alkylthio groups.

2. The compound of claim 1, or a pharmaceutically acceptable salt thereof, wherein Rₐ is selected from the group consisting of C₁-C₃ alkyl, C₁-C₅ alkoxy, cyclopropyl, C₁-C₃ alkoxycarbonyl (4-methylphenyl)sulfonyloxymethyl, mono (C₁-C₅) alkylamino di (C₁-C₅) alkylamino, and carboxamido; the C₁-C₃ alkyl group being substituted with one or more halo, hydroxyl, C₁-C₃ alkoxy and C₁-C₃ alkylcarbonyloxyl groups; and HET is:

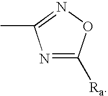

3. The compound of claim 1 or a pharmaceutically acceptable salt thereof, wherein Rₐ is selected from the group consisting of C₁-C₃ alkyl, C₁-C₃ alkoxy, cyclopropyl, C₁-C₃ alkoxycarbonyl, (4-methylphenyl)sulfonyloxymethyl, mono (C₁-C₅) alkylamino, di (C₁-C₅) alkylamino, and carboxamido; the C₁-C₃ alkyl group being substituted with one or more halo, hydroxyl, C₁-C₃ alkoxy and C₁-C₃ alkylcarbonyloxyl groups; and HET is:

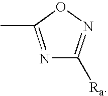

4. The compound of claim 1 or a pharmaceutically acceptable salt thereof, wherein Rₐ is selected from the group consisting of C₁-C₃ allyl, C₁-C₃ alkoxy, cyclopropyl, C₁-C₃ alkoxycarbonyl, (4-methylphenyl)sulfonyloxymethyl, mono (C₁-C₅) alkylamino, di (C₁-C₅) alkylamino, and carboxamido; the C₁-C₃ alkyl group being substituted with one or more halo, hydroxyl, C₁-C₃ alkoxy and C₁-C₃ alkylcarbonyloxyl groups; and is:

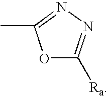

5. The compound of claim 1 or a pharmaceutically acceptable salt thereof, wherein:
Y is alkylene of 3 to 5 carbon atoms; and
Rₐ is selected from the group consisting of monohalomethyl; dihalomethyl; trihalomethyl; 1,1,-dihaloethyl; 1,2 dihaloethyl; 2,2-dihaloethyl; 1,1,2-trihaloethyl; 1,2,2-trihaloethyl and 2,2,2-trihaloethyl.

6. The compound of claim 5 or a pharmaceutically acceptable salt thereof, wherein Rₐ is trifluoromethyl.

7. The compound of claim 6 having the formula: 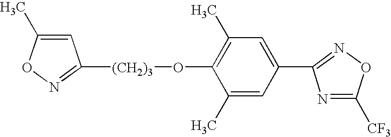
or a pharmaceutically acceptable salt thereof.

8. A composition comprising compound having the formula: 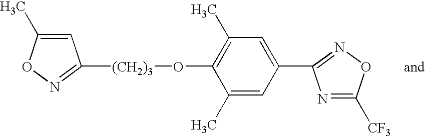
wherein the compound is in isolated or purified form.

9. A composition comprising a compound according to claim 1 or a pharmaceutically acceptable salt thereof.

10. The composition of claim 9 further comprising a pharmaceutically acceptable vehicle.

11. A method for treating or preventing a picornaviral infection in a patient, said method comprising administering to a patient in need of such treatment or prevention a therapeutically effective amount of the compound of claim 1 or a pharmaceutically acceptable salt thereof.

12. A method for inhibiting the growth of a picornaviral infection in a cell said method comprising contacting a cell infected with a picornavirus with an inhibitory amount of the compound of claim 1 or a pharmaceutically acceptable salt thereof.

13. The method of claim 11 wherein the picornavirus is selected from the group consisting of an enterovirus and a rhinovirus.

14. The method of claim 12 wherein the picornavirus is selected from the group consisting of an enterovirus and a rhinovirus.

15. The method of claim 11 wherein the compound of claim 1 has the structure:

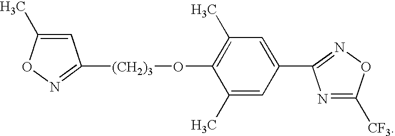

16. The method of claim 12 wherein the compound of claim 1 has the structure.

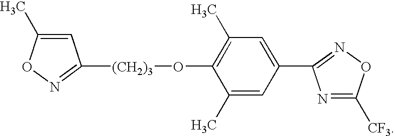

17. The method of claim 15 wherein the compound or pharmaceutically acceptable salt thereof is an isolated or purified form.

18. The method of claim 16 wherein the compound or pharmaceutically acceptable salt thereof is an isolated or purified form.

19. The compound of claim 1, or a pharmaceutically acceptable Salt there, in admixture with a compound of Formula II: 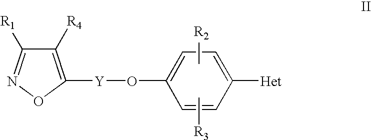
or a pharmaceutically acceptable salt thereof, wherein the R₁, R₂, R₃, R₄, Y, HET and Rₐ groups of the compound of Formula II, or the pharmaceutically acceptable salt thereof, independently have the same meaning as in the compound of Formula I of claim 1, or thee pharmaceutically acceptable salt thereof.

20. A composition comprising a pharmaceutically acceptable vehicle, an antivirally effective amount of a compound of claim 1, or a pharmaceutically acceptable salt thereof, and an antivirally effective amount of a compound of Formula II: 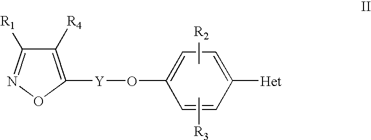
or a pharmaceutically acceptable salt thereof, wherein the R₁, R₂, R₃, R₄, Y, HET and Rₐ groups of the compound of Formula II, or the pharmaceutically acceptable salt thereof, independently have the same meaning as in the compound of Formula I of claim 1, or the pharmaceutically acceptable salt thereof.

21. A pharmaceutical composition comprising a compound of the formula: 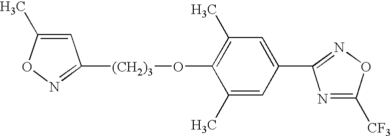
or a pharmaceutically acceptable salt thereof, in admixture with a compound of the formula: 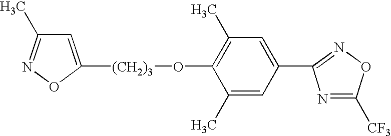
or a pharmaceutically acceptable salt thereof.

22. The composition of claim 20 wherein the compound of claim 1 has the formula: 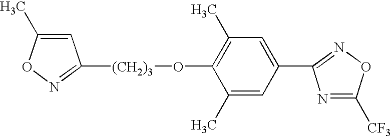
or a pharmaceutically acceptable salt thereof, and the compound of Formula II has the formula: 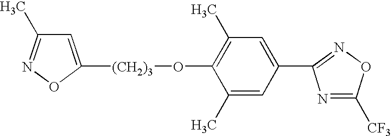
or a pharmaceutically acceptable salt thereof.

23. A method for treating or preventing a picomaviral infection in a patient, said method comprising administering to a patient in need of such a treatment or prevention a therapeutically effective amount of a combination comprising a compound according to claim 1, or a pharmaceutically acceptable salt thereof, and a compound of Formula II: 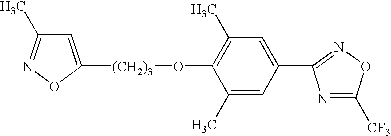
or a pharmaceutically acceptable salt thereof.

24. A method for inhibiting the growth of a picomaviral infection in a cell, said method comprising contacting a cell infected with a picornavirus with a picornavirus Inhibiting amount of a combination comprising a compound of claim 1, or a pharmaceutically acceptable salt, thereof and compound of Formula II: 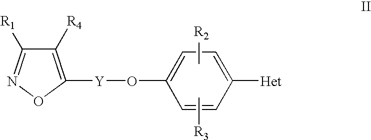
or a pharmaceutically acceptable salt thereof, wherein the R₁, R₂, R₃, R₄, Y, HET and Rₐ groups independently have the same meaning as in the compound of Formula I of claim 1, or the pharmaceutically acceptable salt thereof.

25. The method of claim 23 wherein the compound of claim 1 has the formula: 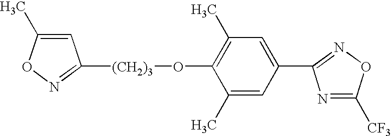
or a pharmaceutically acceptable salt thereof, and the compound of Formula II has the formula: 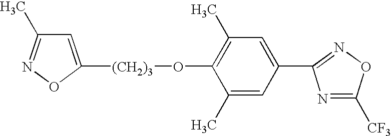
or a pharmaceutically acceptable salt thereof.

26. The method of claim 24 wherein the compound according to claim 1 has the formula: 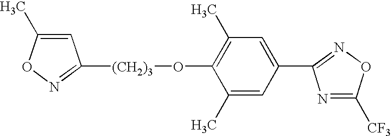
or a pharmaceutically acceptable salt thereof, and the compound of Formula II has the formula: 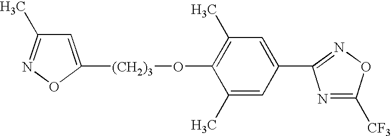
or a pharmaceutically acceptable salt thereof.
